(19) United States
(12) Patent Application Publication
(10) Pub. No.: US 2007/0179617 A1
(43) Pub. Date: Aug. 2, 2007

(54) PROSTHETIC WIDE RANGE MOTION FACETS AND METHODS OF FABRICATING SAME

(75) Inventors: Rebeccah Brown, Decatur, GA (US); Guilhem Denoziere, Atlanta, GA (US); Matthew Miller, Tampa, FL (US)

Correspondence Address: MYERS BIGEL SIBLEY & SAJOVEC, PO BOX 37428, RALEIGH, NC, 27627, US

(73) Assignee: SpineMedica Corporation

(21) Appl. No.: 11/626,399

(22) Filed: Jan. 24, 2007

Related U.S. Application Data
(60) Provisional application No. 60/761,881, filed on Jan. 25, 2006.

(51) Int. Cl. A61F 2/44

(52) U.S. Cl. 623/17.13

(57) ABSTRACT

Facet joint prosthetics have a thin substantially elastomeric body configured to allow movement between adjacent facet joint bones. The facet joint includes bone, cartilage, synovial tissue, and menisci. Related methods of treating a spinal facet joint include inserting a first thin planar elastic body into a spinal facet joint where vertebral bodies meet and may optionally include inserting a second thin planar elastic body into a second spinal facet joint at the same spinal location as the first spinal facet joint thereby inhibiting bioreactive degeneration of a non-treated facet.

### RELATED APPLICATIONS

This application claims priority to U.S. Provisional Application Ser. No. 60/761,881, filed Jan. 25, 2006, the entire contents of the above-referenced documents are hereby incorporated herein by reference as if recited in full herein.

### FIELD OF THE INVENTION

The invention relates to implants and may be particularly relevant to spinal facet joints.

### BACKGROUND OF THE INVENTION

Back pain, particularly low back pain (LBP), remains a common musculoskeletal complaint, with a reported lifetime incidence of 60-90%. Various structures have been incriminated as possible sources of chronic LBP, including the posterior longitudinal ligament, dorsal root ganglia, dura, annular fibers, muscles of the lumbar spine, and the facet joints. The facet joints are more formally termed zygapophyseal joints. Lumbar facet joints are small pairs of joints where vertebrae meet on the back side of the lower/lumbar spine. There are also thoracic and cervical facet joints. The facet joints provide stability to spine by interlocking two vertebrae. Facet joints also allow the spine to bend forward (flexion), bend backward (extension), and twist.

Like other joints, the facet joints consist of bone, cartilage, synovial tissue, and menisci that are rudimentary invaginations of the joint capsule. Prostaglandin, a known inflammatory mediator, which has been associated with pain, can be released from facet joints. See Shin et al., Lumbar Facet Arthropathy, emedicine.com (copyright 2005). The two common treatments for facet joint pain are intra-articular steroid/local anesthetic injection under fluoroscopic guidance and Rf ablation to block all sensory input from the joint.

Despite the above, there remains a need for alternative treatment options for facet joints.

### SUMMARY OF EMBODIMENTS OF THE INVENTION

Embodiments of the invention are directed to surgical interventional treatments of spinal facet joints using implantable materials and/or devices.

Some embodiments provide floating implants while others are attached to one or more sides of the joint. Some “resurface” one or both spinal facet surfaces with a synthetic facet surface.

Some embodiments are directed to facet joint prosthetics. The facet joint prosthetic has a thin elastomeric body.

In some embodiments, the prosthetic can have a thin substantially planar elastic body configured to substantially conform to a shape of an underlying or overlying exposed bone surface of a facet joint. In position, the body is configured to allow motion between adjacent bones.

Related methods of treating a spinal facet joint include inserting a first thin substantially planar elastic body into a spinal facet joint where vertebral bodies meet and may optionally include inserting a second thin planar elastic body into a second spinal facet joint at the same axial spinal location as the first spinal facet joint thereby inhibiting bioreactive degeneration of a non-treated facet. The bodies can be shaped in situ to substantially conform to a shape of a target facet joint surface in the spinal facet joint.

Other embodiments are directed to medical kits for spinal facet treatment. The kits include an implantable prosthetic comprising a member with a flexible elastomeric body for insertion into a spinal facet joint to allow motion between bones forming the joint and a sterile package enclosing the implantable prosthetic.

Other embodiments are directed to spinal facet treatment kits that include a plurality of thin conformable implantable facet bone resurfacing elastic bodies and a sterile package enclosing the elastic bodies.

Still other embodiments are directed to methods of fabricating a spinal facet implant. The methods include: (a) molding an elastomeric primary body of into a desired facet shape; and optionally (b) attaching at least one mesh layer to the implant body so that the at least one mesh layer extends beyond the bounds of the molded implant body to define a bone anchoring segment.

In some embodiments, the mesh layer comprises a polyester mesh material having a thickness that is less than about 1 mm.

Other embodiments are directed to a facet joint prosthesis having a molded freeze-thaw hydrogel core with an outer surface having a partially embedded mesh layer defining a tissue ingrowth scaffold.

Further features, advantages and details of the present invention will be appreciated by those of ordinary skill in the art from a reading of the figures and the detailed description of the embodiments that follow, such description being merely illustrative of the present invention.

### BRIEF DESCRIPTION OF THE DRAWINGS

FIG. 1 is a side view of a spinal facet joint with an implanted facet prosthetic according to embodiments of the present invention.

FIG. 2 is an enlarged side view of a spinal facet joint prosthetic device according to embodiments of the present invention.

FIGS. 3A-3C are top views of different exemplary facet implant shapes according to embodiments of the present invention.

FIG. 4 is a schematic illustration of an exemplary facet joint.

FIG. 5 is a schematic illustration of a facet prosthetic device positioned in the exemplary facet joint shown in FIG. 4 according to some embodiments of the present invention.

FIG. 6 is a schematic illustration of a facet prosthetic device positioned in the exemplary facet joint shown in FIG. 4 according to some embodiments of the present invention.

FIG. 7 is a schematic illustration of a facet prosthetic device positioned in the exemplary facet joint shown in FIG. 4 according to some embodiments of the present invention.

FIG. 8 is a schematic illustration of a facet prosthetic device positioned in the exemplary facet joint shown in FIG. 4 according to some embodiments of the present invention.

FIG. 9 is a schematic illustration of a facet prosthetic device positioned in the exemplary facet joint shown in FIG. 4 according to some embodiments of the present invention.

FIG. 10 is a schematic illustration of a facet prosthetic device positioned in both facet joints according to some embodiments of the present invention.

FIG. 11A is a side view of an alternative embodiment of a facet implant with an attached bone anchor material according to embodiments of the present invention.

FIGS. 11B and 11C are top or bottom views of exemplary configurations of the bone anchor material shown in FIG. 11A.

FIG. 12A is side view of yet another embodiment of a facet implant with an attached bone anchor material according to embodiments of the present invention.

FIGS. 12B and 12C are top or bottom views of exemplary configurations of the bone anchor material shown in FIG. 12A.

FIG. 13A is a schematic side view of the spine and various spinal facet joints with facet implant resurfacing illustrated with respect to either an upper or lower surface according to embodiments of the present invention.

FIG. 13B is a rear view of a treatment site with two facet implants, one in each of a pair of facet joints at a common axial site (such as one of L4-L7), according to some embodiments of the present invention.

FIG. 13C is a side schematic view illustrating different facet implant conformal configurations according to embodiments of the present invention.

FIG. 13D is a top view of a fifth lumbar vertebra with facet implants according to embodiments of the present invention.

FIG. 14 is a schematic top perspective view of a medical kit with a plurality of facet implants according to embodiments of the present invention.

FIG. 15A is a schematic illustration of a natural facet joint and FIGS. 15B and 15C are schematic illustrations of prepared natural facet joints with altered shapes configured to engage respective implants according to embodiments of the invention.

FIG. 16A is a cross-sectional illustration of a double sleeve implant that can be placed on the prepared facet joint shown in FIG. 15B.

FIG. 16B is an enlarged partial view of the device shown in FIG. 16A illustrating a mesh scaffold integrally attached to the implant body according to embodiments of the invention.

FIG. 17 is a cross-sectional illustration of a deformable spacer suitable for placement in the prepared joint shown in FIG. 15C according to embodiments of the present invention.

FIG. 18 is a flow chart of steps that can be used to carryout embodiments of the present invention.

FIG. 19 is a flow chart of steps that can be used to carryout other embodiments of the present invention.

### DETAILED DESCRIPTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity. Broken lines illustrate optional features or operations unless specified otherwise.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a stricture or feature that is disposed “adjacent” another feature may have portions that overlap or underlie the adjacent feature.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention. The sequence of operations (or steps) is not limited to the order presented in the claims or figures unless specifically indicated otherwise.

The term “spinal facet joint” refers to the location at which vertebral bodies meet at a rear portion of the spine. The shape of facet joints change along the length of the spine. The facet joint includes bone, cartilage, synovial tissue, and menisci. The elastic body is configured to conformably reside on an outer surface of the bone in a manner that allows a relatively wide range of motion between the bones forming the joint. The terms “implant”, “prosthesis” and “prosthetic device” are used interchangeably to describe a medical product that is configured to reside in a target face joint of a mammalian subject (for veterinary or medical (human) applications). A facet implant or prosthesis can be applied to one surface (one side) of the facet joint (the bone is resurfaced by the implant) or to both surfaces of the joint, and/or may reside therebetween as a spacer to compress in response to loads introduced by the cooperating bones at the facet joint and still allow motion therebetween.

The term “wide range motion” refers to substantially natural motion of the bones in the facet joint which typically include the three motions associated with a functional spine unit, flexion/extension, lateral bending, and axial rotation. The motions translate differently in the disc compared to the facets but these motions are a good reference as far as range of motion. A facet joint sees sliding motions (along the joint surface) as well as compression and tension (in which case the facets are not in contact and the load is taken by the ligament only (capsular ligament)). The term “compact” means that the device is small with a low profile and suitable for surgical introduction into the spine. The term “thin” means that the device has a thickness that is less than about 6 mm, typically between about 0.001-3 mm, and may be between about 0.01 mm to about 0.5 mm. The term “conformal” means that the implant material or member is sufficiently flexible to conform to a target structure's shape. The target structure's shape can be either the upper portion of the lower bone or the lower portion of the upper bone (one of the two vertebral bones) that meet at the rear of the spine.

The term “mesh” means any permeable and/or porous flexible material in any form including, for example, knotted, braided, extruded, stamped, knitted, woven or otherwise, and may include a material with a substantially regular foramination pattern and/or irregular foramination patterns.

The term “macropores” refers to apertures having at least about a 1 mm diameter or width size, typically a diameter or width that is between about 1 mm to about 3 mm, and more typically a diameter or width that is between about 1 mm to about 1.5 mm (the width dimension referring to non-circular apertures). The macropores may promote bony through-growth for increased fixation and/or stabilization over time.

FIG. 1 illustrates a facet joint 5 on a rear side of the spine between two vertebral bodes 25, adjacent the spinal disc 26. The facet implant 10 can have a thin, compliant elastic body. The size of the prosthesis implant 10 can vary for different individuals. A typical size of an adult implantable facet is between about 5-20 mm width and/or length, more typically between about 10-15 mm, with a thickness between about 0.5 mm to about 8 mm, and more typically between about 1-5 mm, but each of these dimensions can vary. In some embodiments, the implant 10 can have increased friction on one primary surface for anchoring, with reduced friction on the other primary surface, to allow for motion between the bones of the joint. An exemplary coefficient of friction of some implant materials, such as a freeze-thaw hydrogel like Salubria® against Salubria® with a moist interface, is within the range of between about 0.1 to 0.001, typically about 0.02. The values of the freeze-thaw hydrogel (e.g., Salubria®) against bone can vary depending on a number of factors, such as, for example, quality of bone and/or cartilage if present, presence of mesh or not at device interface, etc.

The implant 10 can be configured as an elastomeric, MRI compatible implant that can substantially conform to a bone surface shape at the facet joint. The elastic implant can have a substantially compliant but sufficiently rigid body so as to be relatively stiff to provide a desired compressive modulus of elasticity. The implant 10 can have a solid elastomeric body with compressive strength that is typically less than about 100 MPa (and typically greater than 1 MPa), with an ultimate strength in tension and/or compression that is generally greater than about 100 kPa. The implant 10 can be configured to withstand a compressive load greater than about 1 MPa while allowing motion between the adjacent articulating bones of the facet joint.

The implant 10 can be made from any suitable elastomer capable of providing the desired shape, elasticity, biocompatibility, coefficient of friction, and strength parameters. The implant 10 can be configured with a single, uniform average durometer material and/or may have non-linear elasticity (i.e., it is not constant). The implant 10 may optionally be configured with a plurality of durometers, such as a dual durometer implant. The implant 10 can be configured to be stiffer in the middle, or stiffer on the outside perimeter. In some embodiments, the implant 10 can be configured to have a continuous stiffness change, instead of two distinct durometers. A lower durometer corresponds to a lower stiffness than the higher durometer area. For example, one region may have a compressive modulus of elasticy that is between about 11-100 MPa while the other region may have a compressive modulus of elasticity that is between 1 -10 MPa.

The implant 10 can have a tangent modulus of elasticity that is about 1-50 MPa, typically between about 1-10 MPa, more typically about 3-5 MPa, and a water content of between about 10-60%, typically about 50%. Suitable compressive Tangential Modulus testing parameters are stated below.

| | |
|---|---|
| Test type: | unconfined compression |
| Fixtures: | flat platens, at least 30 mm diameter |
| Rate: | 25.4 mm/sec to 40% strain |
| Temperature: | room temp (˜22° C.) |
| Bath: | samples stored in saline or water until immediately |
| | before test |
| Samples: | cylinders, 9.8 ± 0.1 mm height, 9.05 ± 0.03 mm diameter |

Compressive Tangential Modulus calculated at 15, 20, and 35% strain

Compressive Tangential Modulus Calculated at 15, 20, and 35% Strain

Some embodiments of the implants 10 can comprise polyturethane, silicone, hydrogels, collagens, hyalurons, proteins and other synthetic polymers that are configured to have a desired range of elastomeric mechanical properties. Hydrogels and collagens can be made with mechanical elasticity values less than 20 MPa and greater than 1.0 MPa. Silicone, polyurethane and some cryogels can be configured to have an ultimate tensile strength greater than about 100 or 200 kiloPascals.

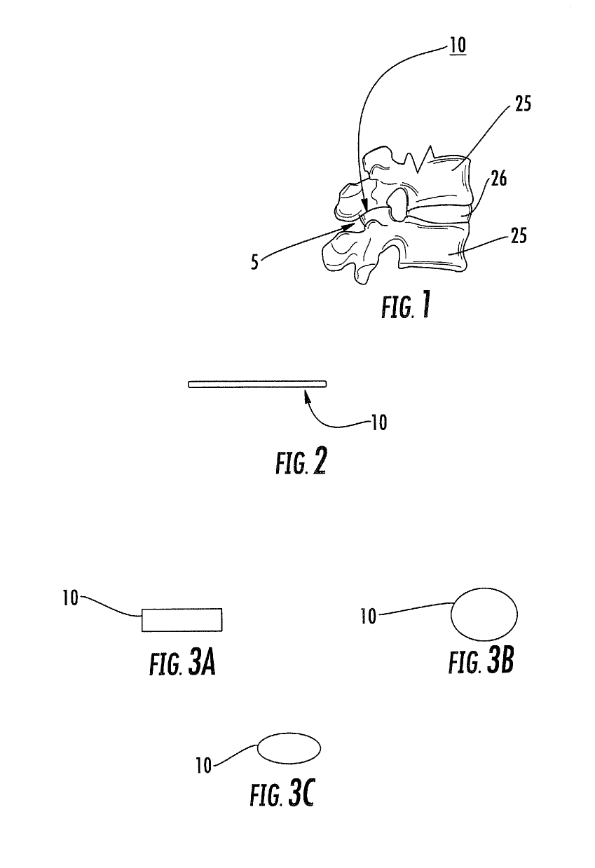

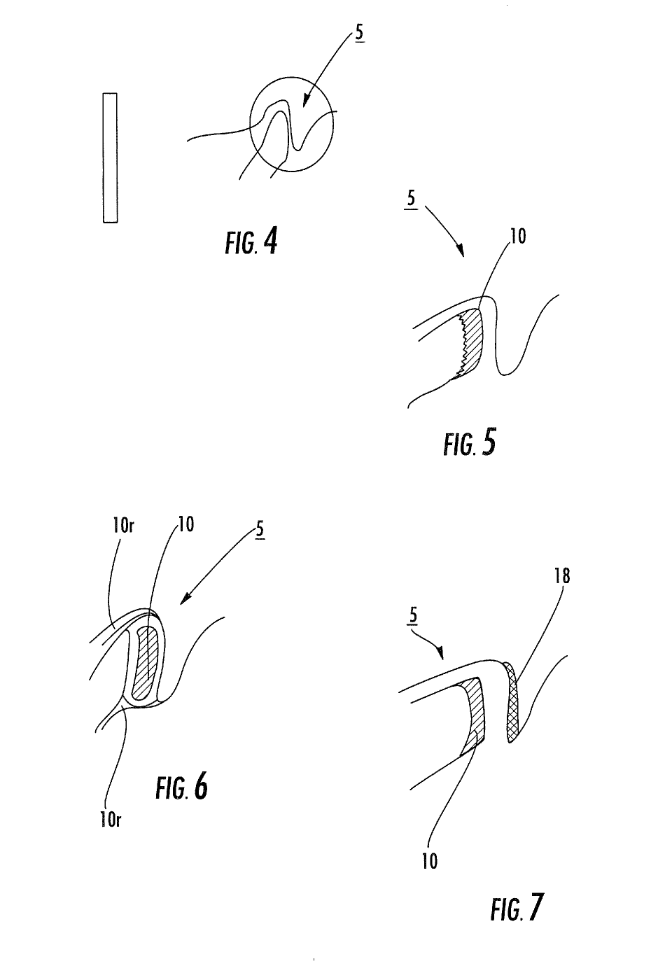

As shown in FIG. 1, a facet joint prosthesis or implant 10 can reside in a target facet joint. As shown in FIG. 2, outside the body, the implant 10 can have a thin, substantially planar shape, which, in some embodiments, can substantially conform to the shape of the underlying surface in situ. FIGS. 3A-3C illustrate that the implant 10 can have different shapes. It is noted that these shapes are exemplary; thus, although shown as substantially rectangular, circular and oval, the implant 10 may have other shapes as well. Typically, the surface area of the implant 10 will be between about 50 mm² and 200 mm², such as between about 10-60 mm², with a thickness of anywhere between about 0.5 to 2 mm. The implant 10 may have a body with a substantially constant thickness or the thickness may vary.

FIG. 4 is a schematic of a spinal facet joint 5. FIG. 5 illustrates the implant 10 attached to one side (either side) of the joint 5 so that one side articulates against the implant 10. The other side can be a natural joint surface or can have a surface that has less compliance resilience than the implant 10 and/or an increased hardness relative to the implant 10. As shown in FIG. 7, the other surface can also have a synthetic surface 18, which may be of the same material as the implant 10, or may comprise metal or a hardened polymer, such as polyethylene (PE). FIG. 6 illustrates that the implant 10 acts as a floating spacer, and is not attached to either side of the joint. Peripheral joint reconstruction using soft tissue, ligaments, or synthetic retainers 10r can hold the implant in the joint. FIG. 8 illustrates that the implant 10 can be attached to both sides of the joint. The implant 10 itself is sufficiently flexible to allow a suitable range of motion between the bones. FIG. 9 illustrates that each side of the joint may have an attached implant 10, and an inner member 19 can be sandwiched therebetween. The inner member 19 may be attached to one or both of the implants 10 or may float, trapped therebetween. In some embodiments, the implants 10 can include an outer lip or outwardly extending edge portion that traps the inner member therebetween to help retain the inner member 19 in position.

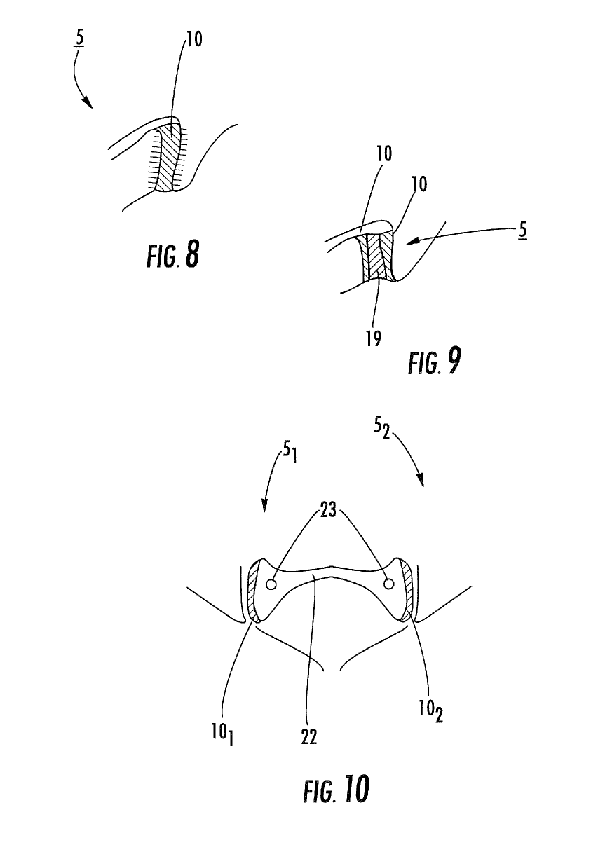

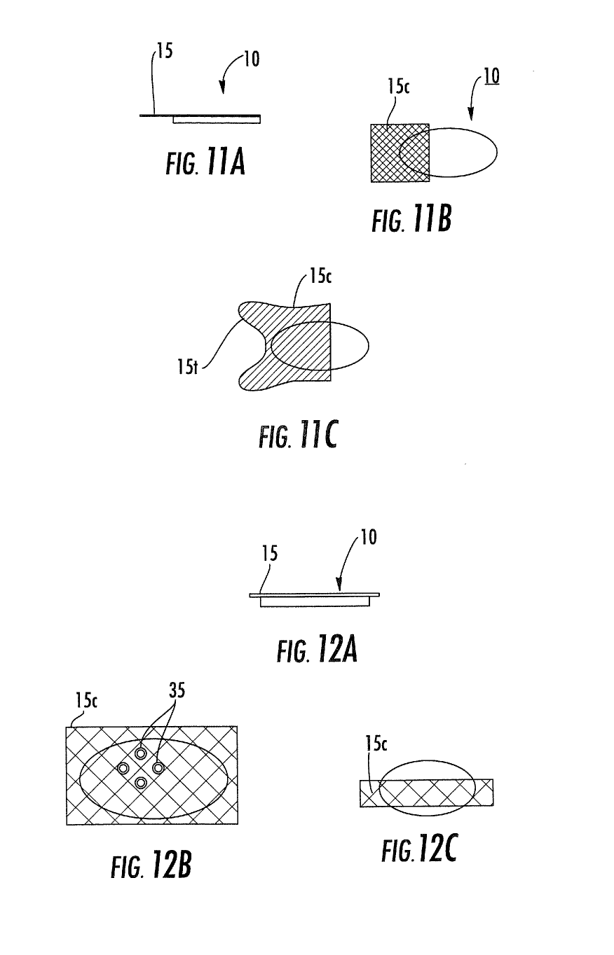

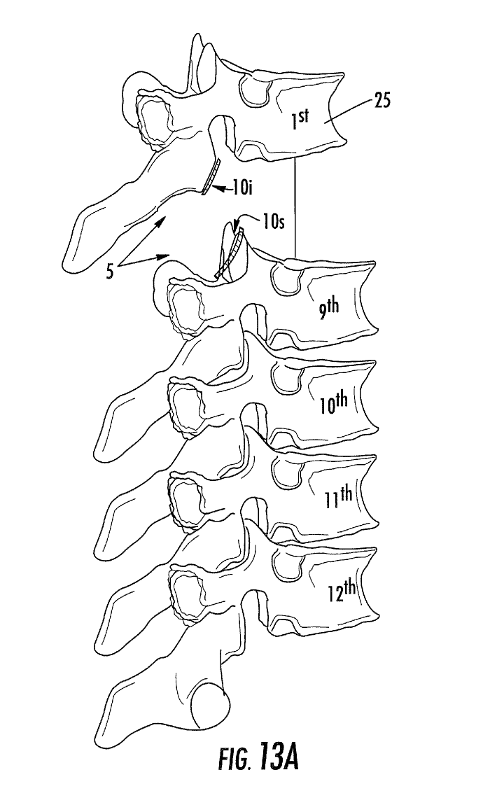

FIG. 10 illustrates that two implants 10₁, 10₂ can be inserted into respective facet joints of facet joint pairs 5₁, 5₂ at a common axial site. In this embodiment, a bridging member 22 can connect each of the portions of the implant 10₁, 10₂ that reside in the respective facet joint itself. The bridging member 22 can attach to either of the upper or lower vertebral bodies. The bridging member 22 can have increased rigidity relative to the portion of the device that resides in the facet joints and may attach to lamina bone using attachment holes 23. The other surface of the facet may also be resurfaced as described above with respect to one or more of the other embodiments. In still other embodiments, the device can be provided as a lamina bone replacement rather than attaching to the bone.

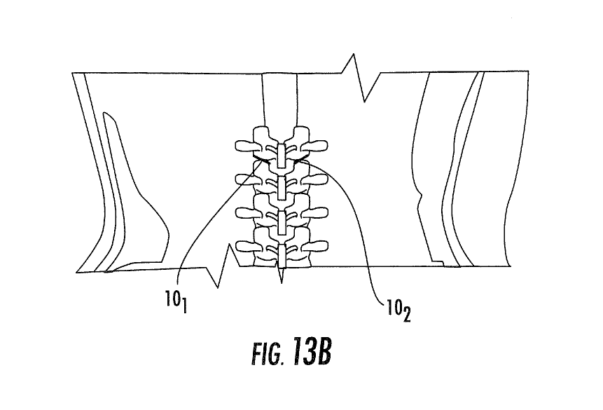

FIG. 11A is a side view of an implant 10 illustrating that the implant 10 can include at least one integral bone anchor segment 15 attached thereto. The bone anchor segment 15 can be malleable or flexible to allow for wrapping over an edge of the facet joint in situ to attach the facet implant to the bone, which has be resurfaced with the implant 10. FIGS. 11B and 11C illustrate that the bone anchor segment 15 can be provided by a cover layer 15c that extends beyond the bounds of the implant body 10.

The cover layer 15c may be an integrally attached mesh (elastomeric, polymer, and/or fabric) layer, such as a polyester fiber mesh, to allow for tissue in-growth as well as local fixation, such as screws or sutures to be inserted therethrough. The cover 15c can have any suitable thickness; typically the thickness is between about 0.001 mm to about 3 mm, typically between about 0.01 mm to about 1 mm. The cover or tabs 15c, 15t can be attached via any suitable biocompatible attachment means, such as heat-sealed, ultrasonically attached, molded, adhesively bonded or stitched together. In addition, in some embodiments, the attachment material 15 may comprise a biocompatible coating or additional material that can increase the stiffness. The stiffening coating or material can include a PVA cryogel. One embodiment of a mesh is a polyester mesh, such as DACRON mesh, having a thickness of about 0.7 mm thick, such as that available as Fablok Mills Mesh #9464 from Fablok Mills, Inc., located in Murray Hill, N.J.

FIG. 11C illustrates that the mesh layer can have a curvilinear perimeter that defines bone anchor attachment tabs 15t. Bone screws 50 or other attachment mechanisms such as sutures and staples can extend through these tabs 15t to help hold the implant in position in the patient's body. FIGS. 11B and 11C illustrate that the bone anchor segments and/or material layer 15c need not extend from the entire perimeter or opposing ends of the device 10. Each tab 15t may be configured as a curved segment as shown in FIG. 11C. The tabs 15t can have a height between about a 2 mm-15 mm with a length (side-side width) being typically between about 2-10 mm. The tabs 15t can be located at various locations about the periphery of the implant (the tabs are typically disposed about either the top or bottom surface). The tabs 15t can be provided by a single continuous skirt, or provided as discrete tab segments.

FIG. 12A illustrates that the implant 10 can have a cover layer 10 that extends beyond opposing sides or ends. FIG. 12B illustrates that the cover layer 10 can extend beyond the entire perimeter of the body 10, while FIG. 12C illustrates that the material layer 15c may only extend beyond two opposing ends. The extension length of the material is sufficient to allow a bone anchor (screw, staple, suture) to be inserted therethrough. In other embodiments, the cover 15c may only cover a portion of the implant primary surface and reside inward of the perimeter (not shown). In some embodiments, as shown in FIG. 12B, the cover 15c can include a plurality of spaced apart apertures 35. The apertures 35 can be macropores that can facilitate bone or tissue in-growth. The apertures 35 can be aligned or misaligned over the implant 10.

In some particular embodiments, the implant 10 can be used without any external covering materials or tabs (not shown). In some embodiments, where attachment is desired, the device 10 may be self-anchoring, adhesively attached, glued or otherwise anchored to the bone without requiring bone anchors extending outside the bounds of the primary body. For example, the bone attachment primary side of the implant 10 can include barbs or anti-migration spikes that extend into bone (not shown). Combinations of the attachment mechanisms described herein can also be used (i.e., barbs and mesh covers or tabs).

In some embodiments, the implant 10 is configured so that the contact surface defined by one primary surface of the implant directly contacts the other bone in the bone pair of the facet joint, with the cover and/or bone attachment segments 15c, 15 only being attached to the other primary surface of the implant. Thus, the cover or tabs 15c, 15t can reside on a single primary surface of the implant 10. Indeed, in some embodiments, a tissue-growth inhibitor may be applied to one or more contact surfaces to facilitate free movement of the bone in the facet joint. In other embodiments, for example, where the cover 15c has suitable frictional, lubricious and/or contact surface properties, or where attachment to both bones is desired, both primary surfaces may include the cover material.

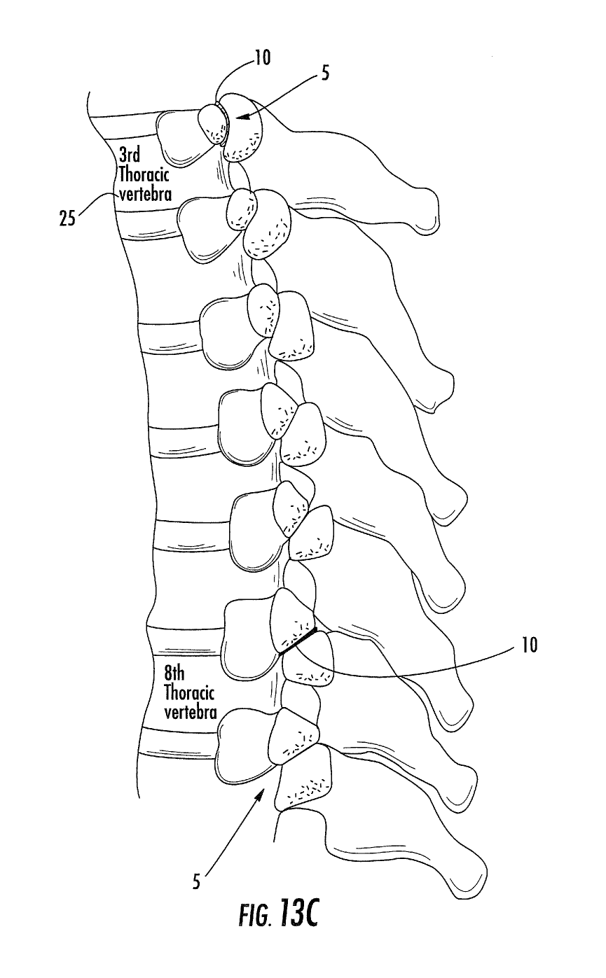

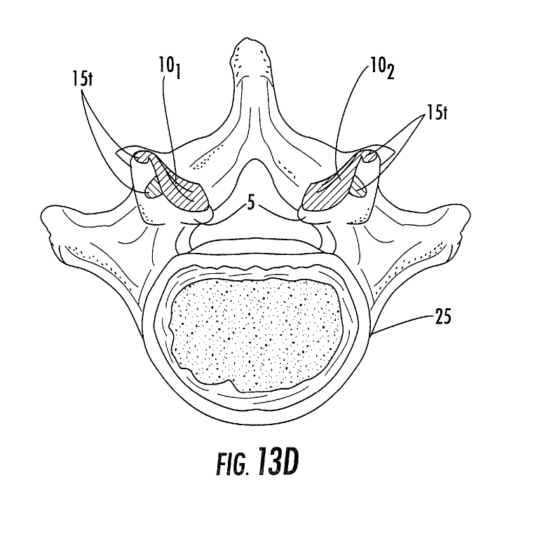

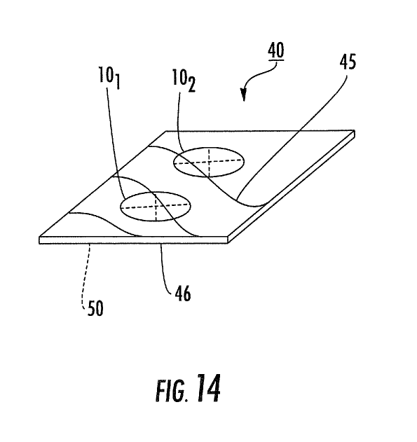

As shown in FIG. 13A, the implants 10 can be fabricated to be used either as a superior facet implant 10s or an inferior facet implant 10i. During a surgical procedure, a clinician can orient the implant 10 to place the cover 15c or bone attachment segment 15 on the target inferior or superior surface, depending on where attachment is desired. In some embodiments, the cover 15c is on a single surface and the physician can orient to place the cover side against the target resurfacing bone.

The bone may be prepared before placing the implant thereon. For example, the target surface can be gently scraped or made to bleed while maintaining the shape of the bone to promote a bioresponse to facilitate a tissue attachment process. The physician can press the implant against the bone surface to conform the shape to the surface. A pressing tool may be used or the forming can be carried out using finger pressure.

FIG. 13B illustrates that the treatment can include inserting two implants 10₁, 10₂, to resurface or insert the prosthesis into both spinal facet joints at a common axial spinal location during a single surgical intervention. This protocol may be used even if only one facet joint is presently indicating a need for treatment in order to help maintain parity in the structure and inhibit deterioration in the other facet joint.

FIG. 13C illustrates the changing shape of the facet joints over the length of the spine. That is, as shown, at T3 the joint 5 is generally vertical and concave, while at T8, it is more horizontal and generally planar. It is contemplated that in some embodiments the implant 10 can be provided in a range of predetermined sizes to allow a clinician to choose an appropriate size for the patient. That is, the implant 10 can be provided in at least two different sizes with substantially the same shape or with different shapes fit to the specific target facet joint/bone. In some embodiments, the implant 10 can be provided in small, medium and large sizes. Further, the sizes can be configured according to the implant position where each or some of the implants may have different sizes—i.e., T2, C, or and L3-L4 implants may have a different sizes from L4-L5 implants. In some embodiments, an implant can be customized (sized) for each respective patient.

In other embodiments, the implant 10 can be configured to be used in and/or to resurface several or most of the spinal facet joint bones. It will be appreciated even if an implant 10 has a first shape, it may be able to have a different shape depending on where it is applied and how malleable the implant is. For example, if applied to a bone at the T3 facet joint, the shape may be different than if applied to, for example, the facet joint at T8.

Similarly, in some embodiments the same implant 10 can be applied either to an inferior or superior surface, and depending which one it is attached to, it can have a different shape, depending on the target location. In some embodiments, the implant can have a substantially concave shape while in others it may be convex or planar.

FIG. 13D illustrates a fifth lumbar vertebra from above. As shown, two implants 10₁, 10₂ have been applied to the facet joint 5. In this embodiment, tabs 15t have been wrapped over an edge of the facet surface to allow for bone anchors to attach thereto (such as bone screws, sutures and/or staples).

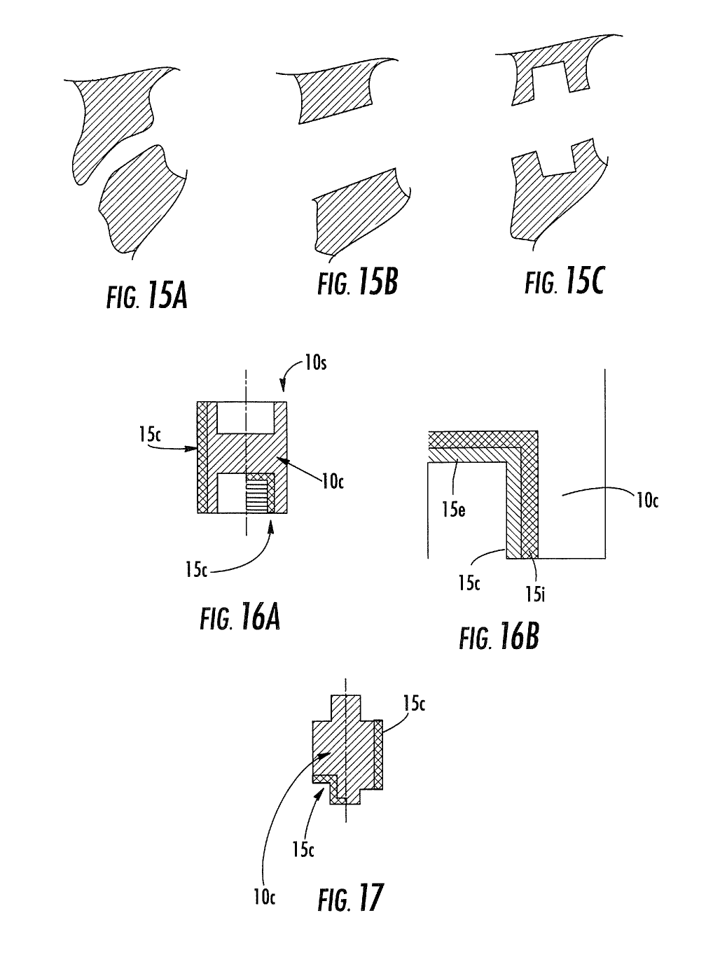

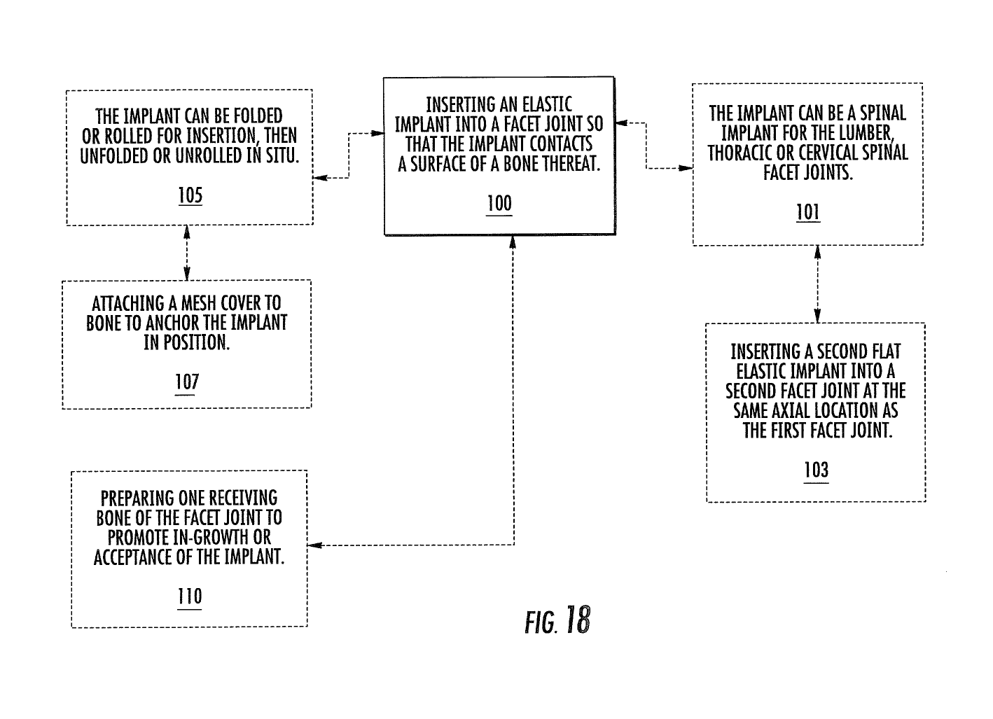

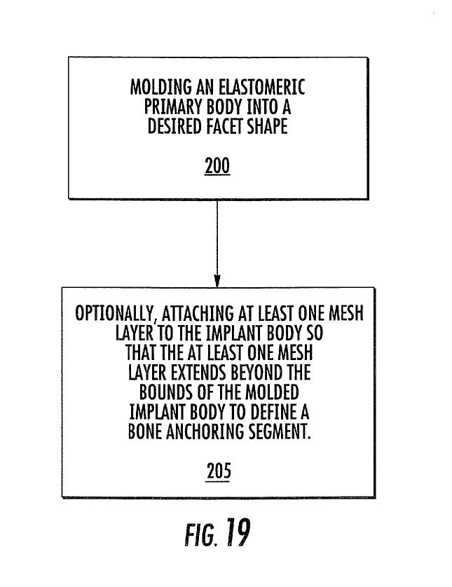

FIGS. 16A and 17 illustrate deformable flexible implants 10 that can deform to restore support and motion of the facet joint 5 as an alternative to using an articulating device. The implant 10 may also request a partial or total facetectomy (removal of the facet joint) as shown in FIGS. 15B, 15C relative to a natural facet joint (FIG. 15A) to allow for more space for the device 10 and associated local fixation means. The implant shown in FIG. 16A can matably receive the prepared joint shown in FIG. 15B while the implant 10 shown in FIG. 17 can be positioned in the prepared joint shown in FIG. 15C. Each implant 10 can have a substantially cylindrical elastomeric solid hydrogel core 10c with a deformable scaffold cover 15c (mesh scaffold) integrally attached thereto. The scaffold cover 15c can be configured to allow for tissue ingrowth and/or local fixation. The scaffold cover 15c can include a tubular mesh on an axially extending outer surface of the implant 10. The scaffold cover 15c may extend over substantially the entire perimeter of the device or about selected portions. As shown in FIGS. 16A, the implant 10 has a double sleeve configuration 10s and the implant in FIG. 17 has a spacer configuration. Each implant 10 can have mesh scaffolding 15c that extends over an outer axially extending outer surface as well as transversely about the facet joint surface contact portions.

FIG. 16B illustrates that the mesh 15c can be partially embedded with the molded material (e.g., hydrogel) to define exposed regions of mesh to at least a partial thickness that can allow for tissue ingrowth. Thus, the mesh 15c can be configured with a double integration fixation configuration, such that at least a major portion of one primary surface can be moldably attached and integrally affixed 15i to the molded core 10c and at least portions of the other opposing primary surface can have exposed regions 15e for fixation in situ. See, e.g., co-pending, co-assigned U.S. Provisional Application Ser. No. 60/885,682, entitled, Methods and Systems for Forming Implants With Selectively Exposed Mesh For Fixation and Related Implants, the contents of which are hereby incorporated by reference as if recited in full herein.

It is noted that the sleeve 10s (FIG. 16A) does not have to be a double sleeve, but can be a single sleeve that resides on one side of the joint, like a protective cup (not shown). The sleeve 10s can also include structural reinforcement and exposed double-integrated scaffold for fixation. In some embodiments, the implant 10 may define an articulating device.

FIG. 14 is a schematic illustration of a medical kit 40 with a plurality of implants 10 (shown as two 10₁, 10₂) in a sterile package 45. The implants 10 may also be individually packaged in sterilized pouches or containers for use. As shown, the implants 10 may be held on a supporting substrate 46 to inhibit shipping or handling damage.

As shown in FIG. 14, the implants 10 may optionally include one or more radiopaque markers, such as radiopaque ink (which may be tungsten based) printed on the cover 15c (mesh section) to allow for easier viewing in medical images. The radiopaque marker(s) may include indicia to allow a clinician to see if the center of the implant has migrated over time (shown in broken line as an alignment cross). In other embodiments, the material itself may be configured to be radiopaque.

FIG. 18 illustrates that an elastic implant can be inserted into a facet joint so that the implant contacts (and may optionally conform to) a target surface of a bone thereat (block 100). As noted above, the implant may be a spinal implant for one of the lumbar, thoracic or cervical spinal joints (block 101). Optionally, a second flat elastic implant can be inserted into a second facet joint at the same axial location as the first facet joint (block 103).

During insertion, the implant 10 may be rolled or folded, then unfolded or unrolled in situ (block 105). In other embodiments, the implant can be introduced in a final “use” form or configuration. A mesh cover extending from the implant can be attached to bone to anchor the implant in position (block 107). One receiving surface of the facet joint can be surgically prepared to promote in-growth or local attachment to the implant while preserving motion in the joint (block 110).

In some embodiments, the implant 10 can include a drug in the body 10 or in the cover 15c to treat pain, promote bone attachment to the implant (osteoconductive salts), and/or inhibit inflammation (anti-inflammatory drugs) due to the surgical intervention.

The implant 10 can be fabricated in any suitable manner, such as, for example, extruded, cut, stamped, and/or molded.

Elastomers useful in the practice of the invention include silicone rubber, polyurethane, polyvinyl alcohol (PVA) hydrogels, polyvinyl pyrrolidone, poly HEMA, HYPAN™ and Salubria® biomaterial. Methods for preparation of these polymers and copolymers are well known to the art. Examples of known processes for fabricating elastomeric cryogel material are described in U.S. Pat. Nos. 5,981,826 and 6,231,605, the contents of which are hereby incorporated by reference. See also, Peppas, Poly (vinyl alcohol) hydrogels prepared by freezing-thawing cyclic processing. Polymer, v. 33, pp. 3932-3936 (1992); Shauna R. Stauffer and Nikolaos A. Peppas.

In some embodiments, the implant 10 comprises a solid crystalline hydrogel body, which can be configured to substantially have its final form before implantation. For example, in some embodiments, the weight percentage of PVA used to form the implant body 10 and the hydration thereof is such that the body 10 has limited expansion once in position in the body. The implant 10 can be configured to have less than 5% expansion in situ, typically less than 1% expansion in situ, and more typically less than about 0.5% expansion in situ. An exemplary hydrogel suitable for forming a spinal facet joint is (highly) hydrolyzed crystalline PVA. PVA cryogels may be prepared from commercially available PVA material, typically comprising powder, crystals or pellets, by any suitable methods known to those of skill in the art. For additional description of suitable methods of producing freeze-thaw hydrogel implants, see co-pending utility applications identified by Attorney Docket No. 9537-5 and 9537-7, and their respective U.S. Provisional Applications 60/761,902 and 60/761,903, the contents of which are hereby incorporated by reference as if recited in full herein.

In some embodiments, the cover 15c (FIGS. 11A, 12A, 16A, 17) can be defined by a material layer integrally (moldably) attached during molding to an outer surface of the implant body. However, a mesh or other anchoring material can be molded subsurface and/or embedded a distance into the implant body and anchored therein to extend a desired distance beyond the bounds of the implant body. The mesh may be locally exposed to a partial or whole thickness to remove the molded material or inhibit the mold material from embedding therein to promote tissue ingrowth as discussed above.

In other embodiments, the mesh can be ultrasonically welded, staked, brazed, adhesively attached, screwed, nailed or otherwise affixed, attached and/or mounted to the implant 10. In some-embodiments, the mesh can be formed using non-elastomeric or non-polymer biocompatible materials including malleable metals, metallic mesh and/or non-porous materials. For non-porous materials, the macropores can be arranged to provide for bone-in growth as needed.

FIG. 19 is a flow chart of exemplary operations that can be carried out to form a facet implant according to embodiments of the present invention. Although described herein as particularly suitable for spinal facet joint implants, it is contemplated that the methods of fabricating implants can be used for other implants as well. As shown, an elastomeric primary body of an implant can be molded into a desired facet implant shape having a defined compressive modulus of elasticity (block 200).

In some embodiments, at least one mesh layer can be attached to the molded implant so that the at least one mesh layer extends outwardly form at least one of a superior or inferior surface of the molded implant to form a bone anchoring segment (block 205).

In some embodiments, to mold the implant, a moldable material comprising an irrigant and/or solvent and between about 20 to 70%, typically between about 25 to 60% (by weight) PVA powder crystals can be placed in a mold having the desired implant shape. The PVA powder crystals can have a MW of between about 124,000 to about 165,000, with about a 99.3-100% hydrolysis. The irrigant or solvent can be a solution of about 0.9% sodium chloride. The PVA crystals can be placed in the mold before the irrigant (no pre-mixing is required). The mold can be evacuated or otherwise processed to remove air bubbles from the interior cavity. For example, the irrigant can be overfilled such that when the lid is placed on (clamped or secured to) the mold, the excess liquid is forced out, thereby removing air bubbles. In other embodiments, a vacuum can be in fluid communication with the mold cavity to lower the pressure in the chamber and remove the air bubbles. The PVA crystals and irrigant can be mixed once in the mold before and/or after the lid is closed. Alternatively, the mixing can occur naturally without active mechanical action during the heating process.

The irrigant and PVA crystals in the mold are heated. Typically, the mold with the moldable material is heated to a temperature of between about 80° C. to about 200° C. for a time sufficient to form a solid molded body. The temperature of the mold can be measured on an external surface. The mold can be heated to at least about 80-200° C. for at least about 5 minutes, typically between about 10 minutes to 4 hours. The temperature can be measured in several external mold locations. The mold can also be placed in an oven and held in the oven for a desired time sufficient to bring the mold and the moldable material to suitable temperatures. The molds can be held in an oven at about 100-200° C. for about 2-6 hours. The higher range may be needed when several molds are placed therein, but different times and temperatures may be used depending on the heat source, such as the oven, the oven temperature, the configuration of the mold, and the number of items being heated.

In some embodiments, osteoconductive material, such as, for example, calcium salt, can be placed on the inner or outer surfaces of the covering layers 15c and/or the inner mold surfaces (ceiling or floor) to coat and/or impregnate the mesh material to provide osteoconductive, tissue-growth promoting coatings. The mold and cover 15c can be configured to provide the bone attachment extension segments discussed above.

After heating, the implant body can be cooled passively or actively and/or frozen and thawed a plurality of times until a solid crystalline implant is formed with the desired mechanical properties. The molded implant body can be removed from the mold prior to the freezing and thawing or the freezing and thawing can be carried out with the implant in the mold. Alternatively, some of the freeze and thaw steps (such as about 2-4 cycles) can be carried out while the implant is in the mold, then others (such as between about 5-15 cycles) can be carried out with the implant out of the mold. The implants 10 can be sterilized with sterile heated liquid or with radiation or other sterilization methods, typically after packaging in medical pouch or other suitable container to provide a sterile medical product.

After freezing and thawing, the molded implant 10 can be placed in water or saline (or both or, in some embodiments, neither during subsequent processing). The device 10 can be partially or completely dehydrated for implantation, but is typically in its final hydrated form to inhibit passive growth in situ. The implant can be a single solid elastomeric material that is biocompatible by cytotoxicity and sensitivity testing specified by ISO (ISO 10993-5 1999: Biological evaluation of medical devices—Part 5: Tests for in vitro cytotoxicity and ISO 10993-10 2002: Biological Evaluation of medical devices-Part 10: Tests for irritation and delayed-type hypersensitivity.).

In some embodiments, the mold can be configured with resilient members such as springs (leaf springs or disc springs) can be inserted underneath one or more screw heads used to attach the mold lid to the mold body. The springs can allow limited or controlled expansion of the mold cavity while keeping the mold closed (retaining the cavity under pressure) to compensate for volume changes as the mold and the molded material therein cool down (the thermal coefficient of the mold and the molded material is typically different). Other thermal compensation mechanisms and configurations may also be used.

Although described primarily herein for spinal facet joints, the facet joint prosthesis implant may also be suitable for facet joints in other body locations such as, for example, the patella, the foot, the arm, fingers, wrists, shoulders and the like.

The foregoing is illustrative of the present invention and is not to limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

That which is claimed:

1. A facet prosthesis, comprising:
a thin elastically deformable elastomeric body configured to reside in a facet joint, wherein, in position, the elastomeric body is configured to allow motion between adjacent bones of the facet joint.

2. A facet prosthesis according to claim 1, wherein the prosthesis is a lumbar, thoracic or cervical facet joint prosthesis.

3. A facet prosthesis according to claim 2, wherein the elastomeric body is sized and configured to attach to one bone joint surface of a bone pair defined by meeting segments of adjacent spinal vertebral bones on a rear side of the spine and allows wide range motion between the bone pair.

4. A facet prosthesis according to claim 1, wherein the elastomeric body is configured to attach to a single bone joint surface of a bone pair defined by meeting segments of adjacent spinal vertebral bones on a rear side of the spine and allows wide range motion between the bone pair.

5. A facet prosthesis according to claim 1, wherein the elastomeric body is configured to float between bone joint surfaces of a bone pair defined by meeting segments of adjacent spinal vertebral bones on a rear side of the spine and allows wide range motion between the bone pair.

6. A facet prosthesis according to claim 1, wherein the prosthesis comprises a second implantable body that is substantially planar and cooperates with the thin elastomeric body, the second body being sized and configured to attach to one bone joint surface of a bone pair defined by meeting segments of adjacent spinal vertebral bones on a rear side of the spine and allows wide range motion between the bone pair.

7. A facet prosthesis according to claim 1, wherein the thin elastomeric body is a first implant member, the prosthesis further comprising second and third members that cooperate on opposing sides of the first member to trap the first member therebetween in a target spinal facet joint to allow wide range motion between the bone pair.

8. A facet prosthesis according to claim 1, wherein the thin elastomeric body is a first implant body, the prosthesis further comprising a second thin elastomeric implant body and a bridging member spanning between each of the first and second bodies, the bridging member configured to attach to a lamina bone, and wherein the first elastic body resides in a first facet joint and the second elastic body resides in a different second facet joint.

9. A facet prosthesis according to claim 2, wherein the elastomeric body comprises solid crystalline polyvinylalcohol hydrogel.

10. A facet prosthesis according to claim 2, further comprising at least one bone anchor segment extending outwardly beyond the bounds of the elastomeric body.

11. A facet prosthesis according to claim 1, further comprising a mesh covering moldably attached to at least one outer surface of the elastomeric body such that the mesh is at least partially embedded with solid elastmeric material.

12. A facet prosthesis according to claim 11, wherein the mesh covering extends beyond the bounds of the elastic body to define at least one bone anchor segment.

13. A facet prosthesis according to claim 1, wherein the elastomeric body is configured to define contact surface of a first bone in the facet joint, and wherein a second bone of the facet joint is configured to move against the contact surface in a manner substantially similar to a natural joint and variably compress the elastomeric body.

14. A facet prosthesis according to claim 1, wherein the elastomeric body is configured to define a contact superior surface on a lower bone in the facet joint.

15. A facet prosthesis according to claim 1, wherein the elastomeric body is configured to define a contact inferior surface on an upper bone in the facet joint.

16. A method of treating a spinal facet joint, comprising:
inserting a first thin substantially planar elastic body into a spinal facet joint where vertebral bodies meet to allow motion between bones forming the spinal facet joint.

17. A method according to claim 16, further comprising substantially conforming the shape of the elastic body to a target facet joint surface of the spinal facet joint.

18. A method according to claim 16, further comprising inserting a second thin planar elastic body into a second spinal facet joint at the same spinal location as the first spinal facet joint.

19. A method according to claim 16, further comprising:
surgically preparing a load bearing portion of a vertebral bone at the target spinal facet joint to promote tissue growth before the inserting step.

20. A method according to claim 16, further comprising attaching the thin planar body to local bone structure in a manner that allows movement of the adjacent vertebral bones meeting at the facet joint.

21. A spinal facet treatment kit, comprising:
an implantable prosthesis comprising a member with a flexible elastomeric body for insertion into a spinal facet joint to allow substantially normal motion between bones forming the joint; and
a sterile package enclosing the implantable prosthesis therein.

22. A kit according to claim 21, wherein the elastic prosthesis is configured to resurface one of the bone surfaces forming the spinal facet joint.

23. A kit according to claim 21, wherein the elastomeric body is sized and configured to attach to two bone joint surfaces of a bone pair defined by meeting segments of adjacent spinal vertebral bones on a rear side of the spine and allows wide range motion between the bone pair.

24. A kit according to claim 21, wherein the prosthesis elastomeric body is configured to attach to a single bone joint surface of a bone pair defined by meeting segments of adjacent spinal vertebral bones on a rear side of the spine and allows wide range motion between the bone pair.

25. A kit according to claim 21, wherein the elastomeric body is sized and configured to float between bone joint surfaces of a bone pair defined by meeting segments of adjacent spinal vertebral bones on a rear side of the spine and allows wide range motion between the bone pair.

26. A kit according to claim 21, wherein the prosthesis comprises a second implantable body that is cooperates with the elastomeric body, the second body being sized and configured to attach to one bone joint surface of a bone pair defined by meeting segments of adjacent spinal vertebral bones on a rear side of the spine and allows wide range motion between the bone pair.

27. A kit according to claim 21, wherein the elastomeric body is a first implant member, the prosthesis comprising second and third members that cooperate on opposing sides of the first member to trap the first member therebetween in a target spinal facet joint to allow wide range motion between the bone pair.

28. A kit according to claim 21, wherein the elastomeric body is a first thin implant body, the prosthesis comprising a second thin elastomeric implant body and a bridging member spanning between each of the first and second implant bodies, the bridging member configured to attach to a lamina bone, and wherein the first body resides in a first facet joint and the second body resides in a different second facet joint.

29. A kit according to claim 21, wherein the elastomeric body comprises solid crystalline polyvinylalcohol hydrogel with a moldably attached mesh on an outer surface thereof.

30. A method of fabricating a spinal facet implant, comprising:
molding an elastomeric primary body of PVA into a desired facet shape.

31. A method according to claim 30, further comprising molding at least one mesh layer to the implant body.

32. A method according to claim 31, wherein the at least one mesh layer is molded to the implant body defines a tissue ingrowth scaffold.

33. A method according to claim 31, wherein the at least one mesh layer is molded to the implant body so that at least one mesh layer extends beyond the bounds of the molded implant body to define a bone anchoring segment.

34. A method according to claim 31, wherein the mesh layer comprises a polyester mesh material having a thickness that is less than about 1 mm.

35. A facet joint prosthesis comprising a molded freeze-thaw hydrogel core with an outer surface having a partially embedded mesh layer defining a tissue ingrowth scaffold.
